(19) United States
(12) Patent Application Publication
(10) Pub. No.: US 2021/0353899 A1
(43) Pub. Date: Nov. 18, 2021

(54) WATER RESERVOIR FOR A DEVICE FOR GAS HUMIDIFICATION IN LAPAROSCOPY

(71) Applicant: Daniel Schwarz, Berlin (DE)

(72) Inventor: Daniel Schwarz, Berlin (DE)

(21) Appl. No.: 17/284,547

(22) PCT Filed: Oct. 3, 2019
(86) PCT No.: PCT/DE2019/000257

§ 371 Date: Apr. 12, 2021

(30) Foreign Application Priority Data
Oct. 10, 2018 (DE) 10 2018 007 981.4

(51) Int. Cl. A61M 16/16; A61M 16/10; A61M 13/00

(52) U.S. Cl. CPC A61M 16/16; A61M 16/1095; A61M 2205/3653; A61M 13/003; A61M 2205/3368; A61M 16/109

(57) ABSTRACT

The present invention relates to a water reservoir for a device for gas humidification in laparoscopy with a water reservoir for connecting to a gas supply device (insufflator).

The present invention relates to a water reservoir for a device for gas humidification in laparoscopy.

### PRIOR ART

Laparoscopy is a medical intervention, in which the abdomen and the organs therein can be visually inspected. For this purpose, usually, small skin incisions (0.3-2 cm) are made in the abdominal wall, and a trocar is introduced therethrough, which in turn can accommodate an optical device. With the aid of a special endoscope (laparoscope), the abdomen can be inspected. In diagnostic laparoscopy, the abdomen is only visually inspected, in a therapeutic procedure, operative interventions can also be performed.

Usually, at the beginning of the laparoscopy, first, the abdomen is filled with gas, so to provide a pneumoperitoneum. For this purpose, various gases have already been used, such as air, nitrogen, or carbon dioxide (CO₂). The use of carbon dioxide gas has proven particularly successful. It was found that in particular in longer laparoscopic interventions, it is reasonable, on one hand, to heat the introduced gas, and on the other hand, to humidify it. Heating of the gas serves for that the patient is not cooled down, so to avoid a diffuse feeling of pain by the patient, which probably is a consequence of local cooling due to the entry of cold gas. The humidification serves for preventing desiccation of the inner abdomen surfaces, also in order to avoid the cooling down occurring thereby.

For this purpose, in prior art, proposals have already been made. In U.S. Pat. No. 6,068,609, for instance, a device is described that enables heating and humidifying of the gas for the laparoscopy. Herein, a separate chamber is described that is provided with a resistance heating element. In the chamber, there is, in addition, an absorbing material, such as a sponge that can be humidified.

Another embodiment of a device for gas humidification in laparoscopy is, for instance, presented in US 2003/0181857 A1. Herein, there is, in the interior of a gas tube, an absorbent material (wick). When the gas flows from the insufflator through the tube, it is simultaneously humidified. Condition is that the material present in the tube is held uniformly humid during the surgery. For this purpose, the document provides a reservoir with water, which is disposed above the tube. Then, through a tube, water is to be brought on the humidification material.

Further prior art Stand is described in the documents US 2013/0239966 A1, EP 0934091 A1, and DE 19510710 A1.

In the practice, it has been found that the uniform supply with humidity is a fundamental problem of this kind of gas humidification device. Depending on the type of surgery and the gas flow required therefor, either too much or too little water is frequently supplied from the water reservoir.

There is the problem, therefore, for an insufflation device used in laparoscopy, comprising a gas supply device and a gas tube supplying the gas to the patient, wherein the tube may optionally contain a humidification material, to specify a water reservoir, which is able to deliver the contained amount of water uniformly into the flowing gas.

Further, it is desired that the handling of the device is not made difficult for the medical personnel. For this purpose, the invention is to enable that the water reservoir can be refilled during an insufflation in progress. The water reservoir can be arranged either close to the device or at a distance to the patient at the tube set. The advantage of positioning close to the device is that the water reservoir is less interfering during the treatment, the advantage of positioning close to the patient is that less condensation in the tube will occur.

It is, therefore, the object of the present invention to provide a simpler device for heating and humidifying the gas that avoids the above disadvantages.

### Solution of the Object

The solution of this object is achieved by the subject matter of patent claim 1, namely

a device for gas humidification in laparoscopy for connecting to a gas supply device (insufflator), including
a water reservoir (1) in a plastic chamber (2) with an optional first absorbent humidification means (3),
wherein the plastic chamber includes a gas inlet (4) and a gas outlet (5),
wherein the gas inlet is connected directly or through an optional tube (6) to the insufflator and wherein the gas outlet leads to a gas tube (7),
one or more substantially cylindrical humidification means made of a second absorbent material (8),
a gas tube (7), through which gas can be supplied into a patient, characterized by that the one or more substantially cylindrical humidification means (8) made of a second absorbent material with at least one cylindrical front face (9) are in contact with the water reservoir (1), and wherein the other cylindrical front face or the cylinder barrel (10) is positioned in the gas flow.

Preferably, at least one of the humidification means is in connection with a heating element or is wrapped by a heating wire (11). The humidification chamber is provided with an optional refill port (12).

Advantageous improvements are subject matter of the sub-claims.

The core of the present invention is a water-containing chamber that is disposed between the gas supply device (insufflator) and the tube leading to the patient. This chamber may contain water in liquid form. Preferably, the water is stored in a humidification material (e.g., cotton wadding, sponge, etc.). From the chamber, the water is conducted through the capillary effect of a second humidification means (e.g., cotton tamponade) into the gas flow (see FIG. 1). It has been found that the humidity transport through the capillary effect is particularly uniformly. Vaporizing water at the surface of the humidification material is uniformly resupplied, depending on consumption. As a result, a particularly uniform humidification of the gas flow is achieved. Preferably, the second humidification means has a substantially cylindrical shape. It may, however, have a shape different from the cylindrical shape, e.g., as a hexagonal column. The ends of the column or of the cylinder, respectively, may also be rounded.

Depending on the amount of gas to be fed, different amounts of water are consumed. It has been found that it may be an advantage, for insufflators with higher gas volume flows (20-50 l/min.), to provide several connections between chamber and heating unit, for instance, three to four cylindrical humidification means (e.g., tamponades with a diameter of one to two centimeters and a length of 4 to 6 cm).

For insufflators with a lower gas volume flow, one to two such elements may be sufficient.

In many cases, it is sufficient, when the gas flow is supplied through the cylindrical front face of the humidification means (e.g., tamponade), in order to achieve sufficient humidification (not shown in the Figures). In other cases, the gas flow is supplied directly via the cylindrical barrel surface of the cylindrical humidification means, as shown in FIG. 1. Preferably, the tamponade is directly wrapped with heating wire and acts as an additional heating unit.

Optionally, the cylindrical humidification means may be heated, for instance, by included or wrapped around heating wires that are heated by electrical resistance heating. In FIG. 1 is shown the spiral wrapping of a tamponade with a heating wire.

Optionally, the gas tube may also contain a humidification material (e.g., a viscose fleece strip contained in the gas tube). In this case, it is appropriate, when the material is in direct contact with the cylindrical humidification means. In this case, the water rising due to the capillary effect can be conducted over the viscose fleece strip. It is possible to simultaneously provide a heating device in the gas tube that heats the gas conducted through the tube. The heating can simultaneously heat the contained viscose fleece strip and thus secure the heating and humidification of the gas.

In an alternative embodiment, the water reservoir may also be disposed concentrically about the gas flow (FIGS. 1 to 3). For this purpose, for instance, an absorbing material, e.g., cotton, may be disposed in a substantially round chamber, with the gas flow being supplied axially through the core of the chamber. In this case, too, the humidity can be conducted through the contained material of the chamber through cylindrical humidification means (e.g., tamponades) into the gas flow. In this case, the tamponades are disposed, for instance, orthogonally to the gas flow, so that the gas flow is supplied through the outer barrel surface of the cylindrical tamponade. In this case, too, an electrical resistance heating element may be provided that, for instance, is realized by a heating wire spirally wound about the tamponade. In this case, too, the capillary effect of the tamponade provides a particularly uniform humidification of the gas flow. The advantage of this embodiment is that the humidification power is independent of the position of the chamber. Several tamponades may be positioned successively in the gas flow. Here, the tamponades may be disposed in parallel to each other (FIG. 2).

Preferably, however, they are not disposed in parallel to each other, but at an angle a of a=180°: n, wherein n is the number of tamponades: For two tamponades, an angle of 90° has proven optimum (FIG. 3), for three tamponades, an angle of 60° each has proven optimum.

In any case, the chamber is provided with corresponding holding elements for the humidification means.

Of course, the correspondingly designed chamber may contain a refill device, for instance, formed by a stub to be filled by means of a syringe.

It is, of course, apparent to those skilled in the art that the chamber of this humidification device needs not be exactly round, but other embodiments are possible, for instance, square, hexagonal, or octagonal embodiments.

It is common to all embodiments that before starting the laparoscopic interventions, sufficient humidification of all components, i.e., of the humidification material serving as a water reservoir and of the tamponade, has to be secured. The water reservoir according to the invention is usually stored and shipped in dry form. Before starting the surgery, therefore, filling with liquid has to be made. This will normally occur through the filling or refilling stub with a connected septum. Alternatively, the water reservoir only may be humidified, and one will have to wait until the tamponade is completely soaked. It is a matter of course, for those skilled in the art, that the liquid supplied before starting the surgery as well as, if applicable, during the surgery needs to be sterile.

Optionally, there is the possibility to provide the water reservoir and/or the tamponade with a humidity indicator that, for instance, indicates by a color change, when the material is humidified. In this case, a uniform coloring will have to be awaited before starting the surgery.

The water reservoir according to the invention can either be placed immediately at the gas outlet of the insufflator or be attached at the tube that leads to the patient. Usual insufflator models frequently contain, at this gas outlet, a separate filter. In this case, the reservoir can immediately be positioned at the filter and form a continuous housing part for holding the filter and the reservoir. Alternatively, the gas outlet of the insufflator can be connected by a tube to the gas inlet of the reservoir.

The chamber for the water reservoir may be made from conventional materials, which are used in the medical technology (for instance, PVC, PE, PP, etc.), for instance, by injection molding or 3D printing. The chamber or parts thereof can also be made transparent, in order to allow a visual inspection.

At the gas outlet end of the water reservoir, the heating tube is positioned. The latter may immediately be glued or welded. Another possibility is that the gas outlet side of the chamber is formed into a stub, onto which the actual gas tube is fitted and fixed.

The gas tube itself is made in usual fashion of plastic.

In view of the numerous descriptions in prior art, separate explanations are not necessary here.

Of course, it is possible to provide the gas tube with a heating wire that, for instance, secures a uniform heating process of the gas flow over the full length of the heating tube. At the distal (patient-side) end of the gas tube, a connection for the medical instrument is provided, which conducts the gas into the body (e.g., a Veress needle or trocar).

Further, optionally, the gas tube may be provided with a temperature sensor, for instance, at the patient-side end of the gas tube, in order to monitor the resulting gas temperature

### DESCRIPTION OF THE FIGURES

FIG. 1 shows the cross-section of a gas humidification device according to the invention with a substantially cylindrical humidification means (8), which is wrapped by a heating wire (11). The first humidification means is in a round plastic chamber that is disposed concentrically about the gas flow (arrows).

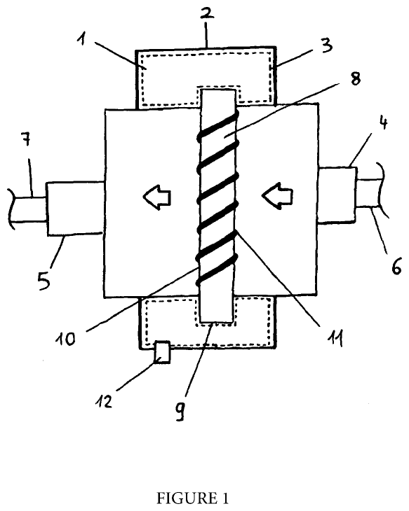

FIG. 2 shows a possible configuration for several (here: three) cylindrical humidification means (8) in the gas flow. The cylindrical humidification means (8) are disposed in parallel in the gas flow in a manner that the gas flow successively passes the cylinders.

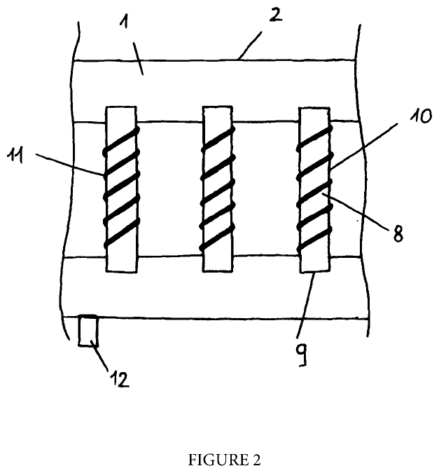

FIG. 3 shows another possible configuration for several (here: two) cylindrical humidification means (8) in the gas flow. The cylindrical humidification means (8) are disposed at the angle of 90° relative to each other.

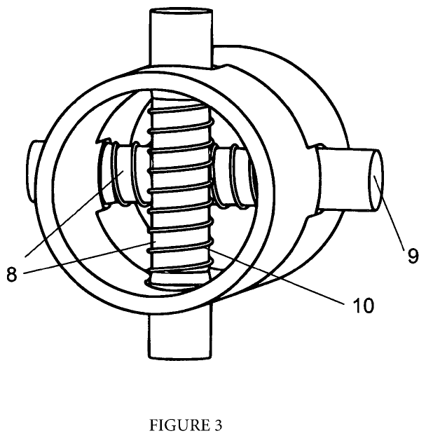

### LIST OF REFERENCES

1) water reservoir,
2) plastic chamber,
3) first absorbent humidification means,
4) gas inlet into the plastic chamber,
5) gas outlet from the plastic chamber,
6) optional tube from insufflator to the gas inlet (4) of the plastic chamber (2),
7) tube from the gas outlet (5) of the plastic chamber (2) to the patient,
8) second absorbent humidification means (substantially cylindrical),
9) cylindrical front face of the substantially cylindrical second humidification means (8),
10) cylindrical barrel surface of the substantially cylindrical second humidification means (8),
11) heating wire,
12) optional access to the plastic chamber (2) for the humidification of the humidification means.

1. A device for gas humidification in laparoscopy for connecting to a gas supply device (insufflator), including
a water reservoir in a plastic chamber with an optional first absorbent humidification means,
wherein the plastic chamber includes a gas inlet and a gas outlet,
wherein the gas inlet is connected directly or through an optional tube to the insufflator, and wherein the gas outlet leads to a gas tube,
one or more substantially cylindrical humidification means made of a second absorbent material,
a gas tube, through which gas can be supplied to a patient,
wherein the one or more substantially cylindrical humidification means made of a second absorbent material with at least one cylindrical front face are in contact with the water reservoir, and wherein the other cylindrical front face or the cylinder barrel is positioned in the gas flow.

2. The device of claim 1, wherein the first and/or the second humidification means is made from a material, which can transport liquid by the capillary effect.

3. The device of claim 1, wherein the plastic chamber contains an access for humidifying the humidification means.

4. The device of claim 1, wherein a heated gas tube at the gas outlet of the plastic chamber.

5. The device of claim 1, wherein a temperature probe in the gas tube.

6. The device of claim 1, wherein the plastic chamber contains one to five substantially cylindrical humidification means.

7. The device of claim 1, wherein the one to five substantially cylindrical humidification means are positioned in the gas flow such that the gas flow is supplied via a cylindrical front face.

8. The device of claim 1, wherein the one to five substantially cylindrical humidification means and/or the first humidification means are heated.

9. The device of claim 1, wherein the water reservoir with the first humidification means is disposed concentrically about the gas flow.

10. The device of claim 1, wherein the gas tube is heated by a heating wire.

11. The device of claim 1, wherein that the one to five substantially cylindrical humidification means are positioned in the gas flow such that the gas flow is supplied via the cylindrical barrel surface.
